(19) United States
(12) Patent Application Publication
(10) Pub. No.: US 2016/0058712 A1
(43) Pub. Date: Mar. 3, 2016

(54) 3-HYDROXYPTEROSTILBENE AND THERAPEUTIC APPLICATIONS THEREOF

(71) Applicants: Muhammed Majeed, Edison, NJ (US); Kalyanam Nagabhushanam, East Windsor, NJ (US); ChiTang Ho, New Brunswick, NJ (US)

(72) Inventors: Muhammed Majeed, Edison, NJ (US); Kalyanam Nagabhushanam, East Windsor, NJ (US); ChiTang Ho, New Brunswick, NJ (US)

(21) Appl. No.: 14/833,321

(22) Filed: Aug. 24, 2015

Related U.S. Application Data
(60) Provisional application No. 62/043,610, filed on Aug. 29, 2014.

(51) Int. Cl. A61K 31/09; G01N 33/50

(52) U.S. Cl. CPC A61K 31/09; G01N 33/5011; G01N 33/5044

(57) ABSTRACT

The invention discloses the therapeutic potential of 3,4-dihydroxy-3′,5′-dimethoxy-trans-stilbene(3-hydroxypterostilbene or 3-HPT) in colon cancer and prostate cancer.

### CROSS-REFERENCE TO RELATED PATENT APPLICATIONS

This application constitutes the non-provisional filing for U.S. provisional patent application 62/043,610 filed on Aug. 29, 2014.

### BACKGROUND OF THE INVENTION

1. Field of Invention

The present invention in general relates to 3,4-dihydroxy-3′,5′-dimethoxy-trans-stilbene. More specifically, the present invention relates to the therapeutic potential of 3,4-dihydroxy-3′,5′-dimethoxy-trans-stilbene in the management of colon and prostate cancer.

2. Description of Prior Art

The anticancer potential of 3,4-dihydroxy-3′,5′-dimethoxy-trans-stilbene(3-hydroxypterostilbene or 3-HPT) has been established for leukemia in M. Tolomeo et al. The International Journal of Biochemistry & Cell Biology, No. 37. 2005. The reference teaches that pterostilbene and 3-HPT are effective apoptosis-inducing agents in multi drug resistant and BCR-ABL-expressing leukemia cells. However, the cross-functional application of 3-HPT across the oncology zone in tackling other forms of cancer cannot be generalized or made obvious from prior art as chemotherapeutic management for cancer varies significantly with specific cancer type and the underlying histological subtypes. For example, Carboplatin and paclitaxel have current widespread acceptance as initial chemotherapy specific for ovarian cancer (Danijela Jelovac et al, CA Cancer J Clin. 2011 May-June; 61(3): 183-203 Robert L. Coleman, “Nat Rev Clin Oncol. 2013 April; 10(4): 211-224). Given the teaching that the process of gathering chemotherapy information has helped to establish specific protocols (types of drugs, doses of drugs and schedule of drugs) based on the type of cancer, stage of cancer, and other specifics about a person's cancer, the evaluation of 3-HPT as an anti-cancer agent assumes critical importance and forms the guiding principle of the current invention (Chemocare, “How do doctors decide which chemotherapy drug to give?”, Available at http://chemocare.com/chemotherapy/what-is-chemotherapy/how-do-doctors-decide-which-chemotherapy-drugs-to-give.aspx).

It is thus the principle objective of the present invention to disclose the therapeutic application of 3-HPT in the management of cancers other than leukemia.

It is also another objective of the present invention to disclose the therapeutic potential of 3-HPT in the management of colon and prostate cancers.

The present invention fulfills the aforesaid objectives and provides further related advantages.

### SUMMARY OF THE INVENTION

The present invention discloses the therapeutic potential of 3-HPT in the management of colon and prostate cancers. More specifically, the present invention discloses the apoptotic and autophagy properties exhibited by 3-HPT in controlling tumorigenesis in the colon and prostate glands. The present invention also discloses the unexpected anti-tumor effects of 3-HPT over pterostilbene in colon cancer and prostate cancer models.

Other features and advantages of the present invention will become apparent from the following more detailed description, taken in conjunction with the accompanying images, which illustrate, by way of example, the principle of the invention.

### BRIEF DESCRIPTION OF THE DRAWINGS

The patent or application filed contains at least one drawing executed in color. Copies of this patent or patent application, publication with color drawing(s) will be provided by the office upon request and payment of the necessary fee.

FIG. 1 is a graphical representation of the 3-(4,5-dimethylthiazol-2-yl)-2,5-diphenyltetrazolium bromide test evaluating the effects of 3-HPT on the % cell viability of PC3 (Human prostate cancer cell line [cell line characteristic of prostatic small cell carcinoma])

FIG. 2 is a photomicrograph (100× under inverted stage microscope equipped with phase contrast) highlighting the morphological changes evident in PC-3 cell lines treated with graded concentrations of 3-HPT (2.5 μM, 5 μM, 10 μM and 20 μM of 3-HPT).

FIGS. 3a and 3b are graphical flow cytometric representations of the apoptotic cell percentage (sub-G1 population of cell cycle) in PC-3 cells treated with 3-HPT for 48 hours.

FIG. 4 shows the photomicrograph of DNA fragmentation induced in PC-3 cells upon treatment with 3-HPT for 48 hours as analyzed by electrophoresis in 2.0% agarose gel.

FIG. 5 shows the Western Blot performed for the expression of Bax (pro-apoptotic protein) and Bcl-XL (anti-apoptotic protein) in the PC-3 cells treated with 10 μM concentration of 3-HPT.

FIG. 6 is a graphical representation of the mitochondrial membrane potential in PC-3 cells that are treated with graded concentrations of 3-HPT (0 μM, 2.5 μM, 5 μM, 10 μM and 20 μM of 3-HPT).

FIGS. 7a, 7b and 7c shows the graphical representations of the caspase-3, caspase-8 and caspase-9 activity in PC-3 cells treated with graded concentrations of 3-HPT (0 μM, 2.5 μM, 5 μM, 10 μM and 20 μM of 3-HPT) for 48 hours (extrinsic and intrinsic apoptotic pathways).

FIG. 8 shows the micrographs of autophagosome formation in PC-3 cells treated 3-HPT and transfected with the green fluorescence protein-LC3 (GFP-LC3)

FIG. 9 shows the Western Blot for autophagy protein expression beclin-1 and LC3-I/LC3-II in PC-3 cells treated with 10 μM 3-HPT over different time intervals in a 48 hour treatment period.

FIG. 10 shows the flow cytometric data generated on the induction of the sub-G1 population of cells as a mark of apoptotic induction, wherein COLO 205 cells were treated with graded concentrations of pterostilbene and 3-HPT (5-100 μM) for 24 hours and the DNA content of the cells were analyzed by flow cytometry.

FIG. 11 shows results of the Western blotting procedure performed on the cell lysates of COLO 205 cells treated with 3-HPT to demonstrate the cleavage of PARP and DFF-45.

FIG. 12 shows the results of the Western blotting procedure performed on the cell lysates of COLO 205 cells treated with 3-HPT to demonstrate the cleavage of pro-caspase 8 and pro-caspase 9.

FIG. 13 show the graphical representation of the kinetics of caspase induction in COLO 205 cells treated with 25 and 50 μM of pterostilbene and 3-HPT for 24 hours.

FIG. 14 shows the flow cytometric data when COLO 205 cells treated with 50 μM of pterostilbene and 3-HPT for 15 minutes were stained with DiOC6 (40 nM) and DCFHDA (20 μM) to measure fluorescence associated with Mitochondrial membrane potential and ROS production.

FIGS. 15a shows the photomicrographs of the generation of acidic vesicular organelles (AVO) by pterostilbene and 3-HPT at 50 μM concentration. Green and red fluorescence in acridine orange stained were observed under the fluorescence microscope. FIG. 15b shows the flow cytometric quantification of autophagy in COLO 205 cells treated with 25 and 50 μM concentration of pterostilbene and 3-HPT for 24 hours and stained with acridine orange followed by measurement of red and green fluorescence.

FIG. 16 shows Western Blotting results for protein LC3 I/II expression in the cell lysates from COLO 205 cells treated with 50 μM pterostilbene and 3-HPT for 24 hours.

FIGS. 17a, 17b and 17c shows the inhibition of mTOR/p70S6K, PI3K/Akt and MAPKs signaling pathways in COLO 205 cells treated with 3-HPT as analyzed by Western Blotting.

FIGS. 18a, 18b and 18c show the inhibition of pterostilbene and 3-hydroxypterostilbene induced autophagy in COLO 205 cancer cells under the influence of autophagy inhibitor chloroquine (CQ).

FIG. 19a shows the photos of tumors induced in the COLO 205 xenograft mice models.

FIG. 19b shows the graphical representation of the reduction in tumor volume in COLO 205 xenograft mice models upon treatment with pterostilbene and 3-HPT at a concentration of 10 mg/kg.

FIG. 19c shows the graphical representation of the reduction in tumor weight in COLO 205 xenograft mice models upon treatment with pterostilbene and 3-HPT at a concentration of 10 mg/kg.

FIG. 19d shows the Western Blot analysis for the protein levels of COX2, MMP9, VEGF, cyclin D1, and procaspase 3 in COLO 205 xenograft tumors from the 10 mg/kg 3-HPT treated group as compared to the pterostilbene group.

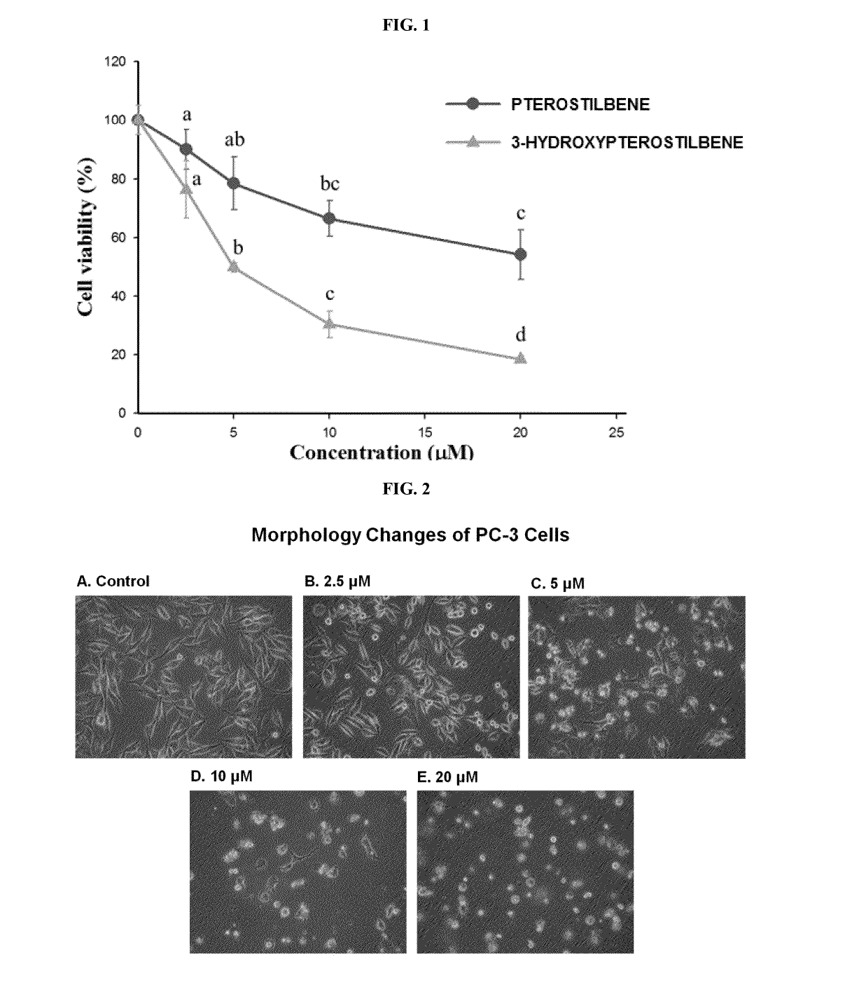

### DESCRIPTION OF THE PREFERRED EMBODIMENTS (FIGS. 1-19)

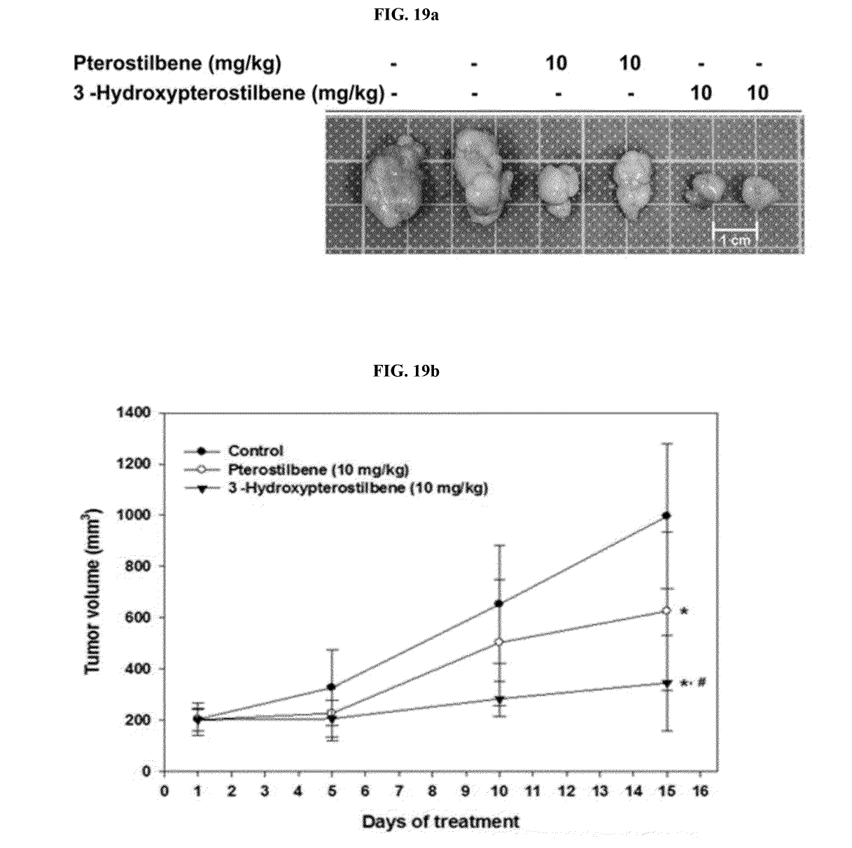

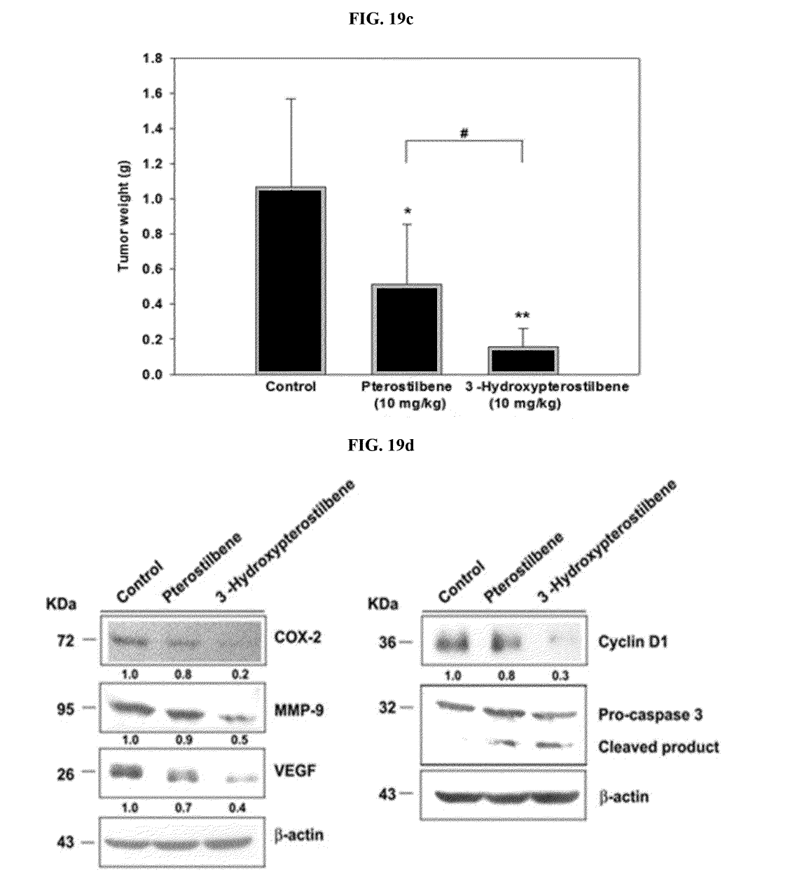

In the most preferred embodiment (FIGS. 19a, 19b, 19c and 19d), the present invention relates to a method of treating mammals suffering from colorectal carcinoma (tumor) by administering 3,4-dihydroxy-3′,5′-dimethoxy-trans-stilbene(3-hydroxypterostilbene) wherein said method comprises step of bringing into contact effective concentration of 3,4-dihydroxy-3′,5′-dimethoxy-trans-stilbene(3-hydroxypterostilbene) and colorectal carcinoma cells to bring about the effects of reduced tumor volume and tumor weight along with down-regulation of tumor promoting proteins. In preferred embodiment, the colorectal carcinoma is human colorectal carcinoma. In another preferred embodiment, the mammal treated is human. In further specific embodiments, the step of administration is one selected from the group consisting of oral administration, intra-peritoneal administration, intra muscular and intra venous administration.

In yet another most preferred embodiment, the present invention relates to 3,4-dihydroxy-3′,5′-dimethoxy-trans-stilbene(3-HPT) for use in the treatment of mammalian colorectal carcinoma. In more specific embodiments, the mammal is human.

In yet another most preferred embodiment, the present invention relates to the use of 3,4-dihydroxy-3′,5′-dimethoxy-trans-stilbene in the manufacture of a medicament for treating mammalian colorectal carcinoma. In more specific embodiments, the mammal is human.

In an alternate embodiment (Table I), the present invention also relates to a method of inhibiting proliferation of human colon cancer cells using 3,4-dihydroxy-3′,5′-dimethoxy-trans-stilbene(3-hydroxypterostilbene), said method comprising steps of
a. Plating human colon cancer cells like COLO 205, HCT-116 and HT-29 at a density of 2×10⁵ cells/mL into 96 well plates;
b. Treating the cells of step a. following overnight growth to series of graded concentrations of 3-hydroxypterostilbene for 24 hours;
c. Adding 0.2% 3(4, 5dimethylthiazol-2-yl)2,5diphenyltetrazolium bromide (MTT) to the cells of step b followed by further incubation of 4 hours: and
d. Measuring cell viability of MTT treated cells of step c using ELISA reader with 570 nm filter.

In another most preferred embodiment, the present invention relates to a method of treating human prostate cancer using effective concentration of 3,4-dihydroxy-3′,5′-dimethoxy-trans-stilbene(3-hydroxypterostilbene) wherein said method comprises step of bringing into contact effective concentration of 3,4-dihydroxy-3′,5′-dimethoxy-trans-stilbene(3-hydroxypterostilbene) and human prostate cancer cell.

In another most preferred embodiment, the present invention relates to 3,4-dihydroxy-3′,5′-dimethoxy-trans-stilbene(3-hydroxypterostilbene) for use in treating mammalian prostate cancer. In more specific embodiments, the mammal is human.

In yet another most preferred embodiment, the present invention relates to the use of 3,4-dihydroxy-3′,5′-dimethoxy-trans-stilbene in the manufacture of a medicament for treating mammalian prostate cancer. In more specific embodiments, the mammal is human.

The anti-colon cancer and anti-prostate cancer properties of 3,4-dihydroxy-3′,5′-dimethoxy-trans-stilbene(3-hydroxypterostilbene) and molecular mechanisms thereof have been highlighted as specific examples in the following sections.

### EXAMPLE I

### Culture of Human Colon Cancer Cells and Assessment of Cell Viability in the Presence of pterostilbene and 3-hydroxypterostilbene

The human colon cancer cell lines COLO 205, HCT116 and HT29 were purchased from the American Type Culture Collection (Rockville, Md.). Cell lines were grown in RPMI1640 supplemented with 10% heat inactivated fetal bovine serum (GIBCO BRL, Grand Island, N.Y.), 100 units/mL of penicillin, 100 μg/mL of streptomycin), 2 mM L-glutamine (GIBCO BRL, Grand Island, N.Y.), and were kept at 37° C. in a humidified 5% CO incubator. Pterostilbene and 3-HPT were dissolved in dimethyl sulfoxide (DMSO, as final concentration of 0.05%). Cells were treated with 0.05% DMSO as vehicle control.

Cell viability was assayed by 3(4,5-dimethylthiazol-2-yl)-2,5-diphenyltetrazolium bromide (MTT). Briefly. COLO 205, HCT116 and HT29 cells were plated at a density of 2×10² cells/mL into 96 well plates. After overnight growth, cells were treated with a series of concentrations of pterostilbene or 3-hydroxypterostilbene for 24 h. The final concentrations of DMSO in the culture medium were <0.05%. At the end of treatment, 0.2% MTT was added and cells were incubated for a further 4 h. Cell viability was determined by scanning with an ELISA reader with a 570 nm filter. The results of the cell cytotoxicity assay are represented in Table I.

TABLE I

Compound IC₅₀ (μM)

| Cell Line | Pterostilbene | 3-hydroxypterostilbene |
|---|---|---|
| COLO205 | 33.4 ± 0.2 | 9.0 ± 0.2 |
| HCT-116 | 47.1 ± 0.6 | 40.2 ± 0.6 |
| HT-29 | 80.6 ± 3.3 | 70.9 ± 1.0 |

### EXAMPLE II

### Flow Cytometry Analysis of subG1 Cell Population, Mitochondrial Membrane Potential and ROS Production

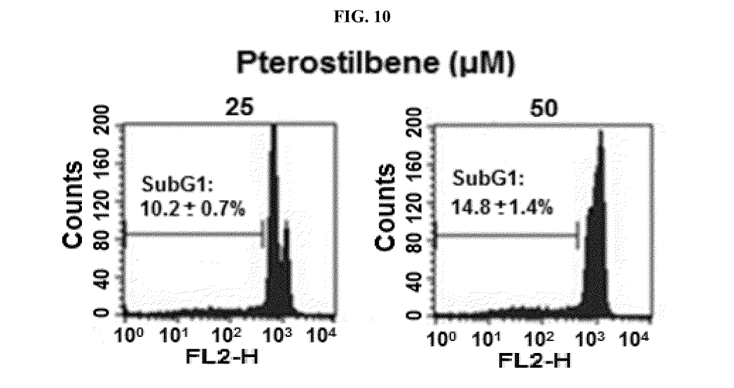

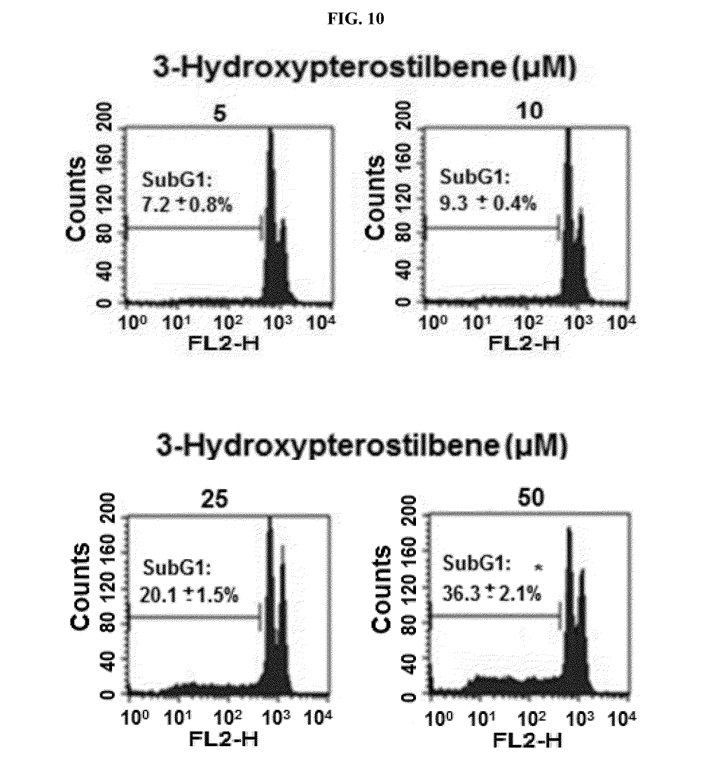

For subG1 cell population analysis, COLO 205 cells (2×10⁵ cells/mL) were cultured in 12 well and treatment with various concentrations of pterostilbene or 3-hydroxypterostilbene for 24 h. The cells were then harvested, washed with PBS, re-suspended in 200 μL of PBS, and fixed in 800 μL of iced 100% ethanol at 20° C. After being left to stand overnight, the cell pellets were collected by centrifugation, re-suspended in 1 mL of hypotonic buffer (0.5% Triton X100 in PBS and 0.5 μg/mL RNase) with PI (50 μg/mL) and incubated at 37° C. in the dark for 30 min. Fluorescence emitted from the PIDNA complex was quantitated after excitation of the fluorescent dye by FACScan cytometry (Becton Dickinson, San Jose, Calif.). Results of the flow cytometry analysis indicated that the percentages of apoptotic COLO 205 cells was 6.2, 8.4, 10.2, 14.8, and 7.2 and 9.3, 20.1 and 36.3% after incubation with 5, 10, 25, and 50 μM pterostilbene and 3-hydroxypterostilbene, respectively (FIG. 10). The results indicated a higher apoptotic potential of 3-hydroxypterostilbene for COLO 205 cells.

### EXAMPLE III

### Effect of 3-hydroxypterostilbene on the Cleavage of PARP (Poly (ADP-ribose) polymerase), DFF-45 (DNA Fragmentation Factor A), Pro-Caspase 8 and Pro-Caspase 9 as Compared to pterostilbene as Analyzed by Western Blotting

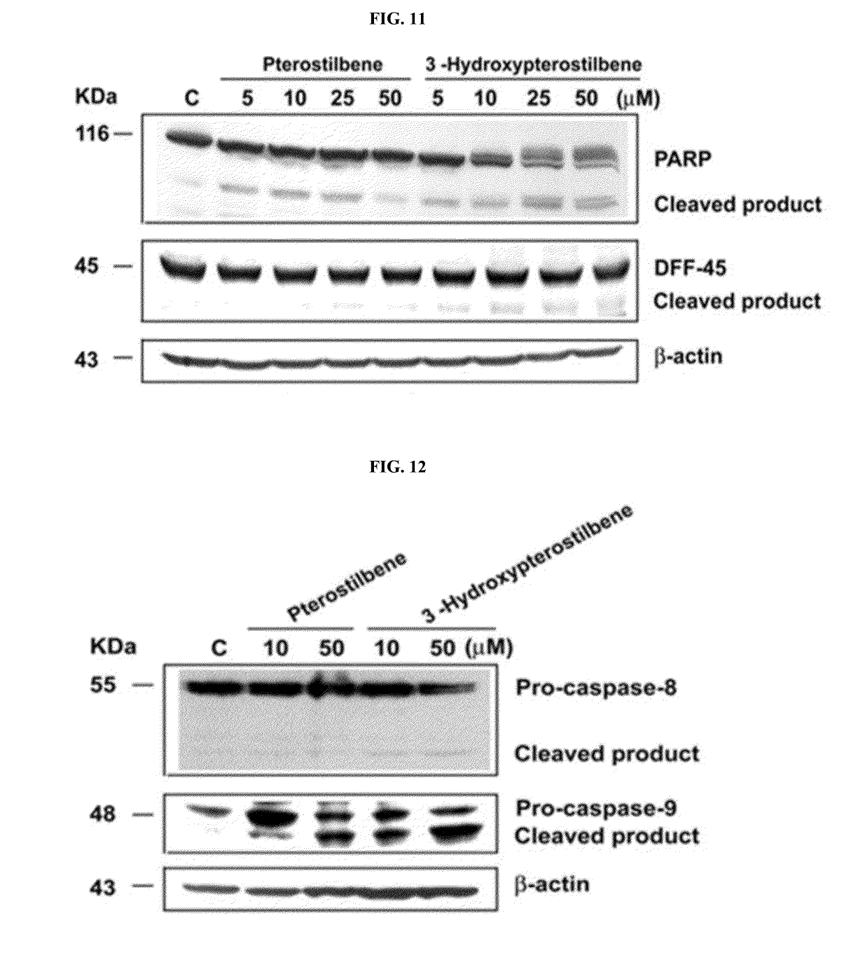

Western blotting analysis was performed as follows. The total proteins of COLO 205 cells were extracted via addition of gold lysis buffer (50 mM TrisHCl, pH 7.4; 1 mM NaF; 150 mM NaCl; 1 mM EGTA; 1 mM phenylmethanesulfonyl fluoride; 1% NP40; and 10 μg/mL leupeptin) to the cell pellets on ice for 30 min, followed by centrifugation at 10,000×g for 30 min at 4° C. The total proteins were measured by BioRad Protein Assay (BioRad Laboratories, Munich, Germany). The samples (50 μg of protein) were mixed with 5× sample buffer containing 0.3 M TrisHCl (pH 6.8), 25% 2mercaptoethanol, 12% sodium dodecyl sulfate (SDS), 25 mM EDTA, 20% glycerol, and 0.1% bromophenol blue. The mixtures were boiled at 100° C. for 5 min and were subjected to 10% SDS polyacrylamide minigels at a constant current of 20 mA. Electrophoresis was then carried out on SDSpolyacrylaniide gels. Proteins on the gel were electrotransferred onto an immobile membrane (PVDF; Millipore Corp., Bedford, Mass.) with transfer buffer composed of 25 mM TrisHCl (pH8.9), 192 mM glycine, and 20% methanol. The membranes were blocked with blocking solution containing 20 mM TrisHCl, and then immunoblotted with different primary antibodies and β actin. The blots were rinsed three times with PBST buffer (0.2% Tween 20 in 1× PBS buffer) for 10 min each. Then blots were incubated with 1:5000 dilution of the horseradish peroxidase (HRP) conjugated secondary antibody (Zymed Laboratories, San Francisco, Calif.) and then washed again three times with PBST buffer. The transferred proteins were visualized with an enhanced chemi-luminescence detection kit (ECL Amersham Pharmacia Biotech, Buckinghamshire, UK). The densities of the bands were quantified with a computer densitometer (AlphaImager™ 2200 System Alpha densitometer. Innotech Corporation, San Leandro, Calif.). The results indicated that as compared with pterostilbene, 3-HPT markedly caused the degradation of 116 kDa PARP into 85 kDa fragments and induced DFF45 protein degradation (FIG. 11). The pro-caspase 9 cleavage by 3-HPT was much better than that exemplified by pterostilbene. The pro-caspase 8 cleavage by 3-HPT was moderate (FIG. 12).

### EXAMPLE IV

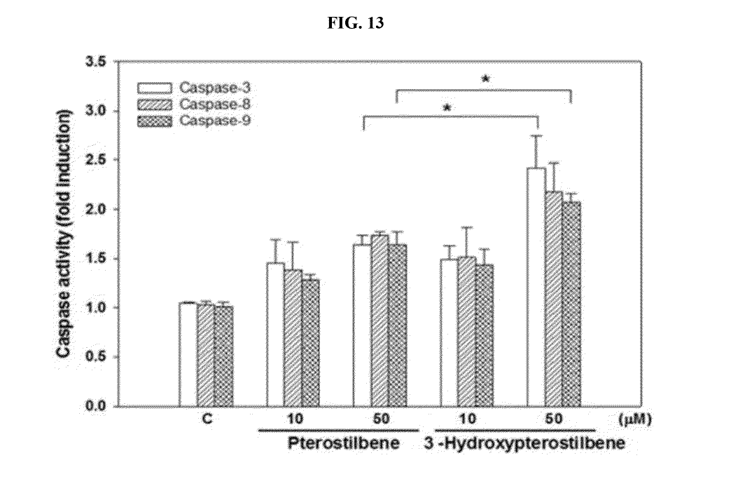

### Caspase 3, 8 and 9 Induction by 3-HPT and pterostilbene (FIG. 13)

The caspase 3, 8 and 9 induction activity in protein extractions of COLO 205 cells was determined by a fluorogenic assay (Promega's CaspACE Assay System, Madison, Wis.). Briefly, 50 μg of total protein, as determined by the BioRad protein assay kit (BioRad Laboratories), was incubated with 50 μM substrate Ac-Asp-Glu-Val-Asp-methylcoumaryl-7-amine (caspase3 specific substrate), Ac-Ile-Glu-Thr-Asp-AMC (Ac-LEND-AMC) (caspase 8 specific substrate), or Ac-Leu-Glu-His-Asp-AMC (Ac-LEHD-AMC) (caspase 9 specific substrate) at 30° C. for 1 h. The release of methylcoumaryl-7-amine was measured by excitation at 360 nm and emission at 460 nm using a fluorescence spectrophotometer (ECLIPSE, Varian, Palo Alto, Calif.). The results indicated that 3-HPT induced a dramatic increase in caspase 3 activity of approximately 2.4 fold after 24 hours of treatment.

### EXAMPLE V

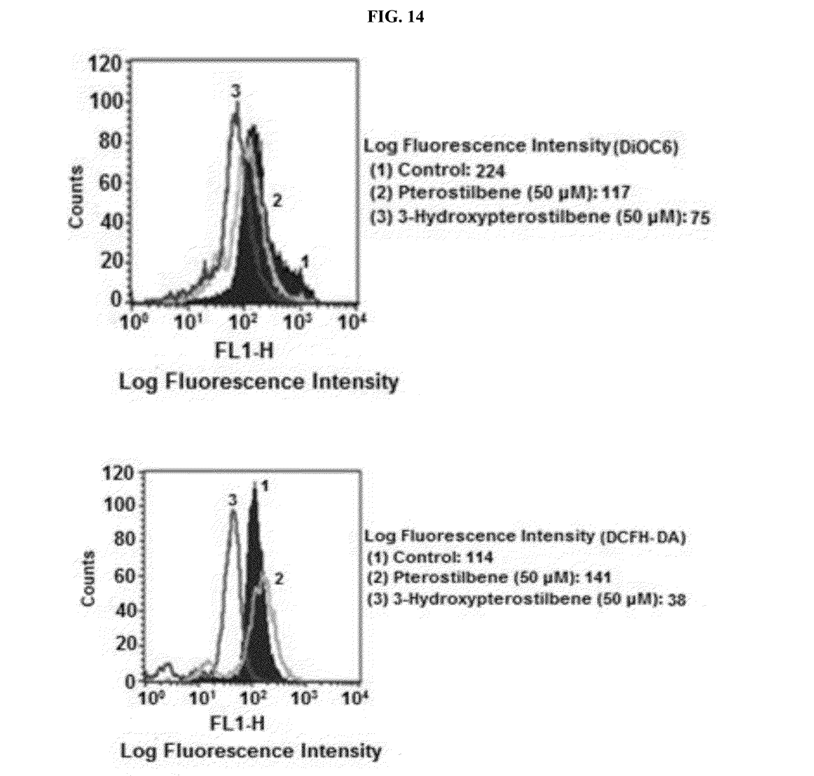

### Effect of 3-HPT and pterostilbene on the Mitochondrial Transmembrane Potential (AT) and ROS (Reactive Oxygen Species) in COLO 205 cells (FIG. 14)

For mitochondrial membrane potential and ROS production, COLO 205 cells were treated as described under Example III for 15 min, and then DiOC6 [3,3′-dihexyloxacarbocyanine iodide] (40 nM) or DCFHDA [2′,7′-dichlorodihydrofluorescein diacetate] (20 μM) was added to the medium for a further 30 min at 37° C. After washing with PBS, the fluorescence intensity was determined by FAC Scan cytometry. The DiOC6 fluorescence intensity shifted to the left from 224 to 117 and 75 in pterostilbene and 3-HPT induced apoptotic COLO 205 cells, respectively. These results confirmed that 3-HPT caused a decrease in the mitochondrial trans membrane potential in COLO 205 cells. The role of ROS in the induction of apoptosis is well recognized. Interestingly, 3-HPT markedly decreased the mean DCFHDA fluorescence intensity from 114 to 38, whereas pterostilbene increased DCFHDA fluorescence intensity from 114 to 141 at 15 min. The imbalance of ROS concentrations could play an important role as an early mediator in 3-HPT induced apoptosis.

### EXAMPLE VI

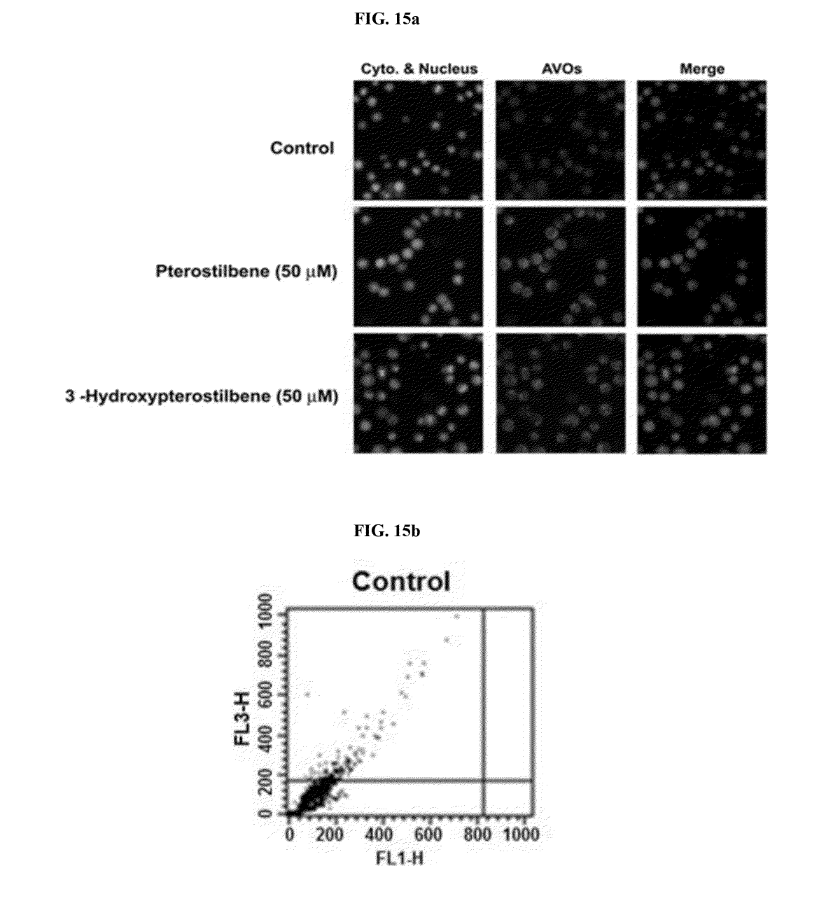

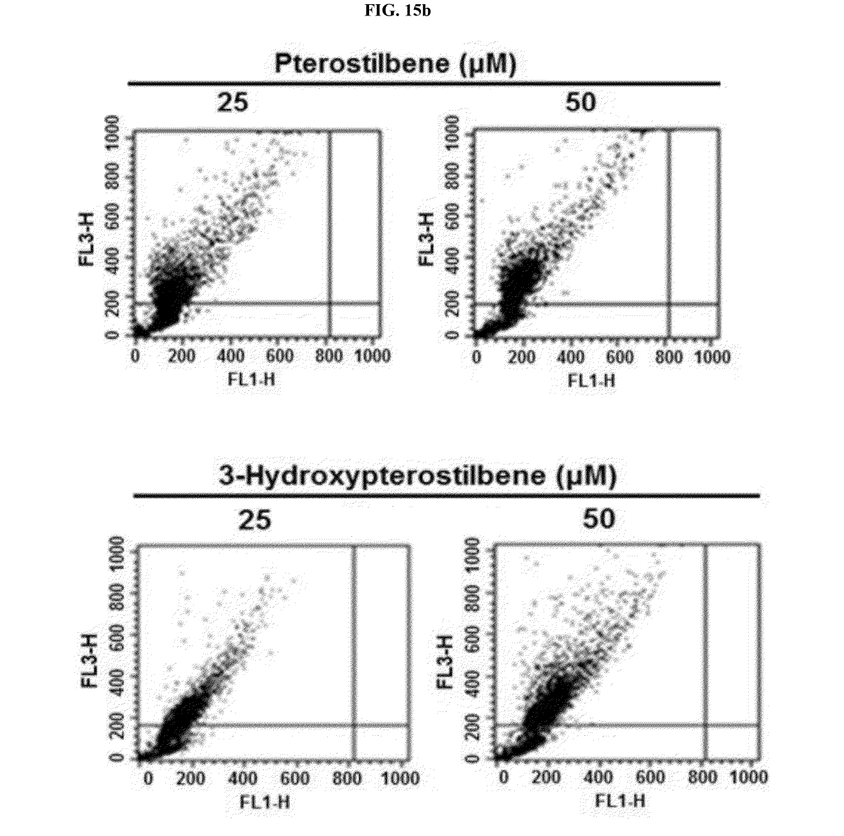

### Autophagy Induction by 3-HPT (FIGS. 15a and 15b)

Autophagy induction in COLO 205 cells treated with 3-HPT and pterostilbene was detected by Acridine Orange (AO) staining. After 24 h treatment, COLO 205 cells were washed with phosphate buffered saline (PBS), suspended in PBS and stained with 1 μg/mL AO of 20 min. Photographs were obtained with a fluorescence microscope (Axioscop, Carl Zeiss, Thomwood, N.Y.) equipped with a mercury 100 W lamp, 490 nm Bandpass blue excitation filters, a 500 nm dichroic mirror and a 515 nm long pass barrier filter. For quantification of AVOs by flow cytometry, cells were treated as described under EXAMPLE II and stained with AO for 15 min and were analyzed by FACScan laser flow cytometer and CellQuest software. The fluorescence microscopy showed that 3-HPT treatment resulted in marked appearance of AVO than pterostilbene when cells were stained with acridine orange after 24 h treatment (FIG. 15a). Cells with AVOs showed enhanced red fluorescence as analyzed by flow that significantly increased after treatment with 3-HPT in a dose dependent manner (FIG. 15b).

### EXAMPLE VII

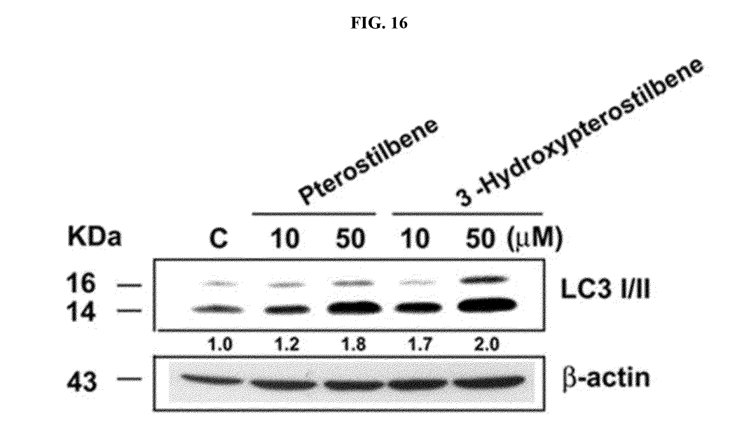

### Expression of LC3 I/II by 3-hydroxypterostilbene Treated COLO 205 Cells (FIG. 16)

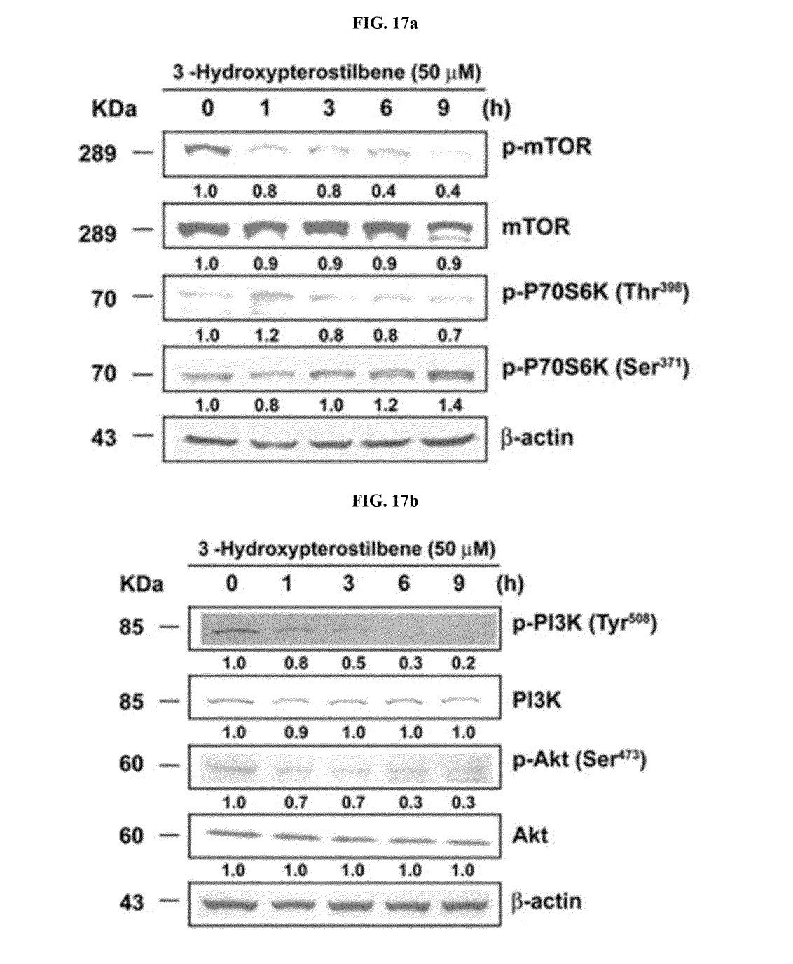

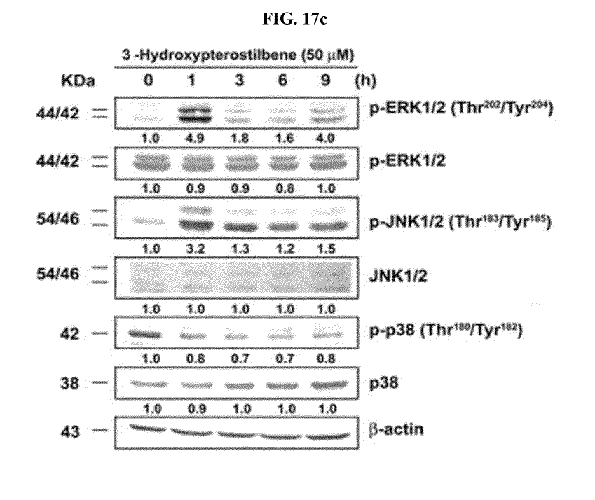

COLO 205 cells were treated with 50 μM pterostilbene or 3-hydroxypterostilbene for 24 hours. Cell lysates were prepared after 24 h treatment and the protein expression of LC3 were analyzed by Western blotting. Enhanced protein LC3 I/II expression in the cell lysates from COLO 205 cells treated with 50 μM 3-hydroxypterostilbene as compared to pterostilbene was observed by the Western Blotting analysis. Western Blotting analysis on COLO 205 cells treated with 50 μM pterostilbene or 3-hydroxypterostilbene for 24 hours also indicated that 3-HPT significantly down regulated the phosphatidylinositol 3 kinase (PI3K)/Akt and mitogen activated protein kinases (MAPKs) signalings including decreased the phosphorylation of mammalian target of rapamycin (mTOR) suggesting that these changes mediate 3-HPT induced autophagy in COLO 205 cells (FIGS. 17a, 17b and 17c).

### EXAMPLE VIII

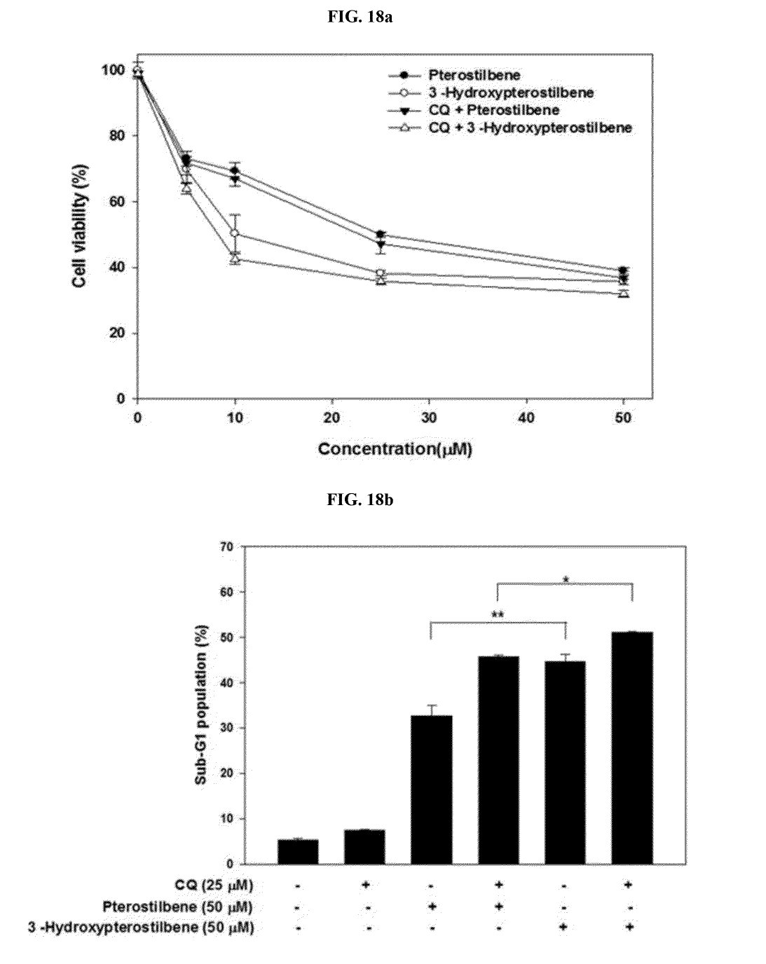

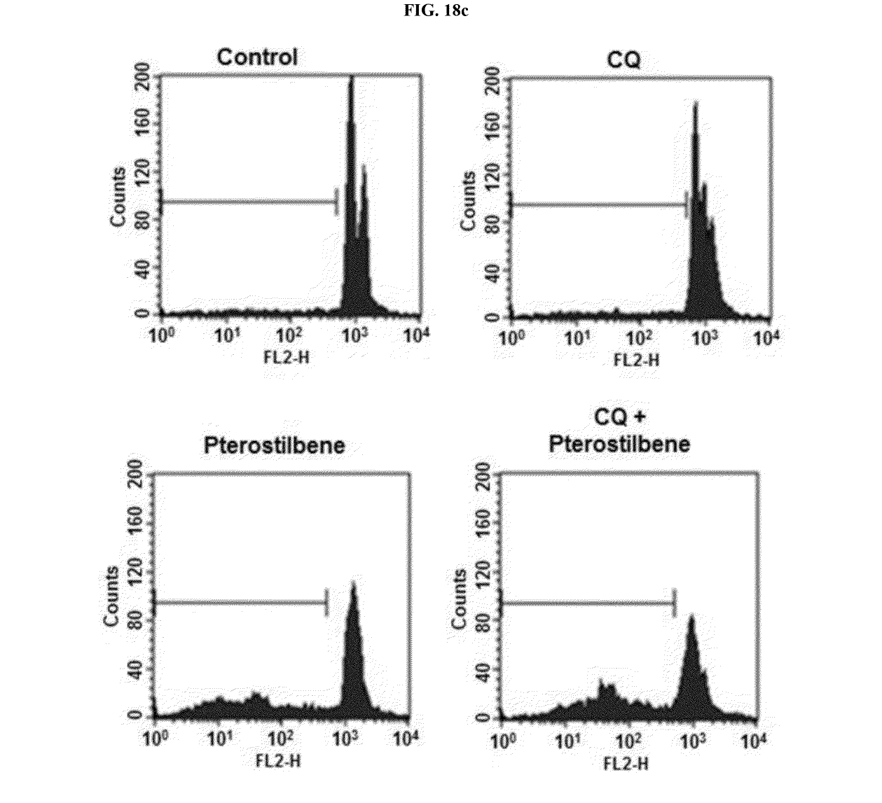

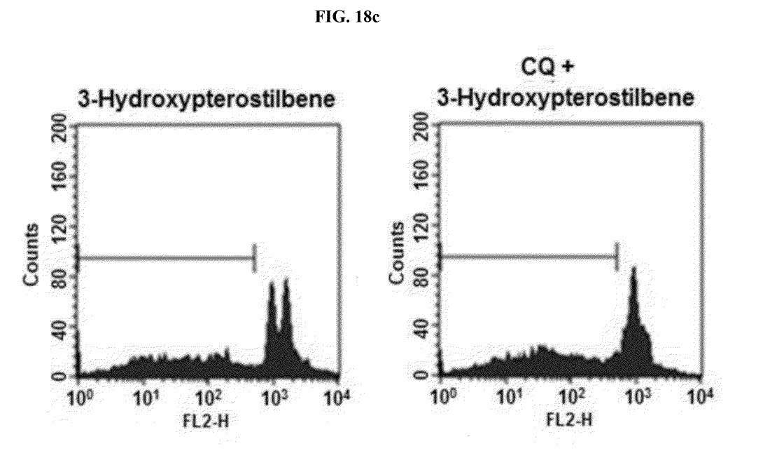

### Suppression of 3-HPT/pterostilbene Induced Autophagy in COLO 205 Cells by Autophagy Inhibitor Chloroquine (CQ) (FIGS. 18a, 18b and 18c)

Cells were pretreated with 25 μM CQ for 1 h before treatment with 50 μM of pterostilbene or 3-hydroxypterostilbene for 24 h. Cell viability was determined by MTT assay. SubG1 cell population (%) was analyzed and quantified after Propidium iodide (PI) staining followed by flow cytometry. The results indicated that that treatment with CQ revealed significant increase cytotoxicity (FIG. 18a) by enhanced 3-HPT triggered apoptosis (FIGS. 18b and 18c). These results corroborated with the observation that 3-HPT treatment induces autophagic cell death in COLO 205 cells.

### EXAMPLE IX

### COLO 205 Xenograft Model

Male Balb/c nude mice at 3-4 weeks old (weighing 16-18 g) were obtained from the BioLASCO Experimental Animal Center (BioLASCO, Taipei, Taiwan). All animals were maintained in pathogen free sterile isolators and in a controlled atmosphere (25±1° C. at 50% relative humidity) and with a 12 h light-12 h dark cycle according to institutional guidelines. Animals were fed with standard AIN76 diet and all food, water, caging, and bedding were sterilized prior to use. Animals had free access to food and water at all times. Food cups were replenished with fresh diet every day. All animal experimental protocol used in this study was approved by Institutional Animal Care and Use Committee of the National Kaohsiung Marine University (IACUC, NKMU, #099AAA902, Validity dates: Jan. 1, 2009 Jul. 31, 2012). After 1 week of acclimation, colon cancer COLO 205 cells (5×10⁶) in 0.2 mL PBS were injected subcutaneously between the scapulae of each nude mouse. After transplantation, tumor size was measured using calipers, and the tumor volume was estimated according to the following formula: tumor volume (mm)=L×W2/2, where L is the length and W is the width. Once tumors reached a mean size of 100-200 mm, mice were randomly divided into three groups (6 animals/group). Mice were i.p. injection with pterostilbene or 3-hydroxypterostilbene (10 mg/kg/d, respectively) for 15 days, while control animals were received injection of corn oil. The diet intake and body weight of each animal was monitored every day. The tumor volume was assessed and recorded every 5 days using caliper measurements. After 15 days, the mice were sacrificed by CO asphyxiation and the liver, kidneys, spleen and solid tumors were excised immediately and weighed. Average tumor volume and tumor weight of each group were represent the mean±standard deviation (SD). Average tumor volumes were recorded during the treatment and average tumor weights were measured at the end of experiment. Six samples were analyzed in each group, and values represent the mean±SD. *P<0.05 and **P<0.01, compared with control group. #P<0.05, compared with pterostilbene treated group. The tumor tissues were cut into several portion for western blot analysis or stored at −80° C. The results of the animal studies showed that
1. The body and organ weights in each group did not show any unhealthy symptoms throughout the course of the study. These results suggested that no noticeable side effects or toxicity were caused by the i.p. injection of pterostilbene and 3-HPT. Furthermore, in mice receiving these treatment regimens, no gross signs of toxicity were observed during visible inspections of general appearance and microscopic examinations of individual organs.
2. After 15 days, tumor volume in 3-HPT treated group was significantly inhibited in comparison with the pterostilbene treated mice (FIGS. 19a and 19b).
3. The tumor weight was strongly inhibited in the 3-HPT treated mice (FIG. 19c).
4. Further, the protein levels of COX2, MMP9, VEGF, cyclin D1, and procaspase 3 were markedly decreased in COLO 205 xenograft tumors from the 10 mg/kg 3-HPT treated group compared to the pterostilbene group (FIG. 19d).

The overall results indicated that 3-HPT was an effective therapeutic agent for colon cancer chemotherapy.

### EXAMPLE X

### Assessment of Cell Viability of Human Prostate Cancer Cells (PC-3) in the Presence of pterostilbene and 3-hydroxypterostilbene

The MTT reaction explained in Para 0040 was also used to assess the cell viability of human prostate cancer cells (PC-3) in the presence of pterostilbene and 3-hydroxypterostilbene. The results (FIG. 1) are represented in Table II.

TABLE II

| | Compound IC₅₀ (μM) | |
|---|---|---|
| Cell Line | Pterostilbene | 3-hydroxypterostilbene |
| PC-3 (Human prostate cancer | >20 μM | 5 μM |
| cell line)-prostatic small cell | | |
| carcinoma | | |

The morphological changes evident in PC-3 cell lines treated with graded concentrations of 3-HPT (2.5 μM, 5 μM, 10 μM and 20 μM of 3-HPT) as viewed microscopically (100× under inverted stage microscope equipped with phase contrast) indicated cell cytotoxicity starting at a concentration of 5 μM 3-HPT (FIG. 2).

### EXAMPLE XI

### Flow Cytometry Analysis of subG1 Cell Population in PC-3 Cells (Analysis of DNA Content) Treated with 3-hydroxypterostilbene

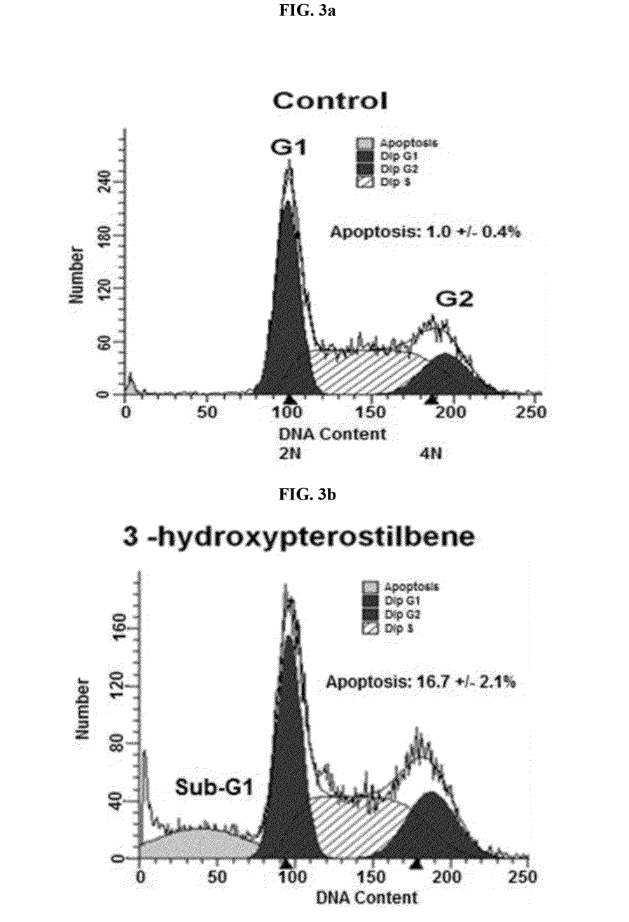

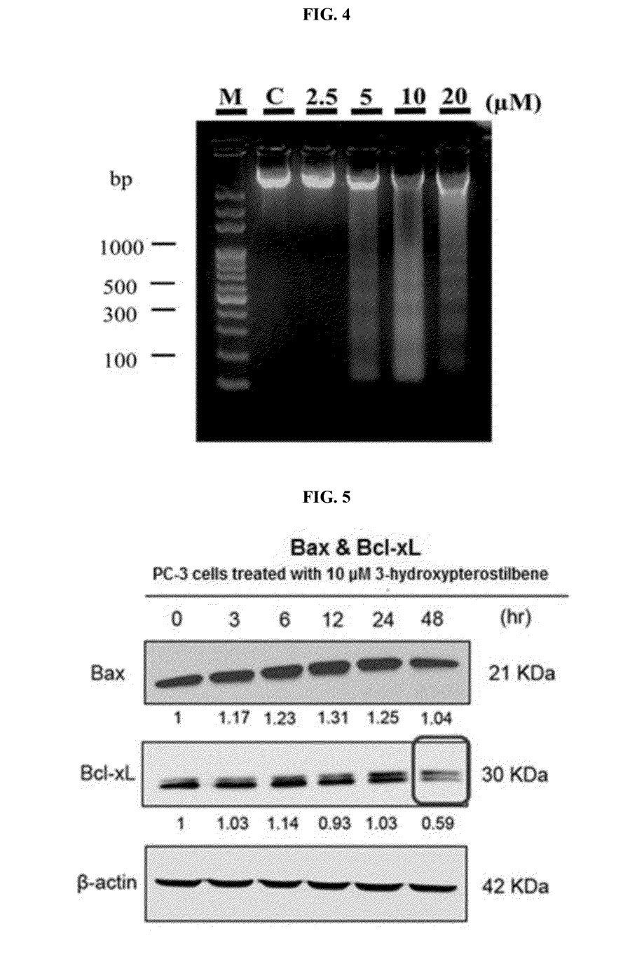

PC-3 cells were cultured in serum-free RPMI containing 0, 2.5, 5, 10, or 20 μM of 3-HPT for 48 hr followed by flow cytometric analysis. The results showed increasing percentage of apoptotic cells over increasing concentrations of 3-HPT (FIGS. 3a and 3b). Further, DNA fragmentation studies on PC-3 cells cultured in serum-free RPMI containing 0, 2.5, 5, 10, or 20 μM of 3-HPT for 48 hr and analyzed by electrophoresis in 2.0% agarose gel indicated apoptotic cells death induced by 3-HPT in PC-3 cells (FIG. 4)

### EXAMPLE XII

### Bcl-xL (Anti-Apoptotic Protein) and Bax (Pro-Apoptotic Protein) in PC-3 Cells Treated with 10 μM of 3-HPT at Different Time Intervals

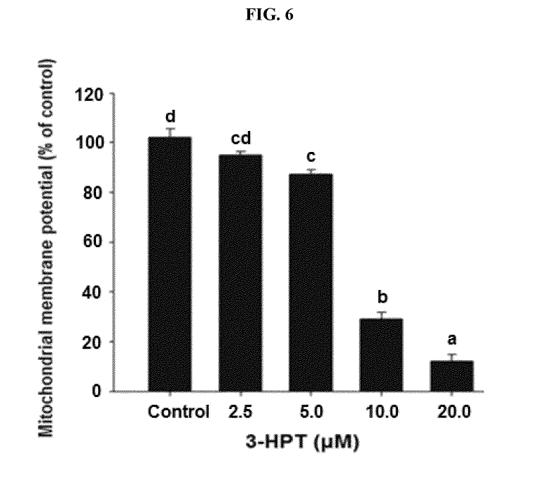

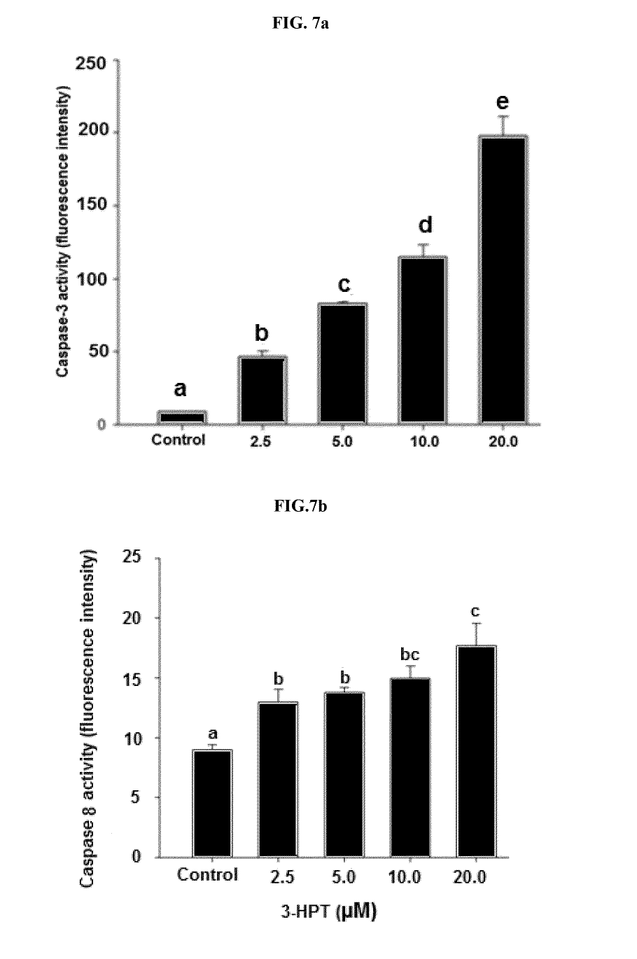

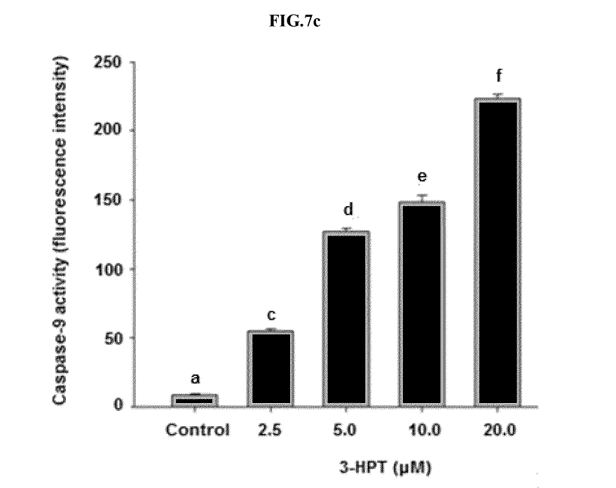

FIG. 5 shows the Western Blot performed for the expression of Bcl-xL (anti-apoptotic protein) and Bax (pro-apoptotic protein) in PC-3 cells treated with 10 μM of 3-HPT at different time intervals. 3-HPT (10 μM) clearly down-regulates Bcl-xL (anti-apoptotic protein) expression in PC-3 cells and upregulates Bax (pro-apoptotic protein) in PC-3 cells. As a measure of down-stream check points in the mammalian apoptotic pathways, the potential of 3-HPT to cause mitochondrial dysfunction and activation of the caspase pathways was further evaluated. PC-3 cells were cultured in serum-free RPMI containing 0, 2.5, 5, 10, or 20 μM of 3-HPT for 48 hr, respectively. Then, cells were stained with DiOC6 and analyzed by flow cytometry. The significant reduction in the mitochondrial membrane potential as seen with 10 and 20 μM of 3-HPT (FIG. 6) indicates initiation of mitochondrial dysfunction. Further, initiation of caspases activity (caspase 3, 8 and 9) as measured fluorometrically is clear evidence of the ability of 3-HPT to activate both the intrinsic and extrinsic apoptosis pathways in PC-3 cells (FIGS. 7a, 7b and 7c)

### EXAMPLE XIII

### Detection of Autophagy

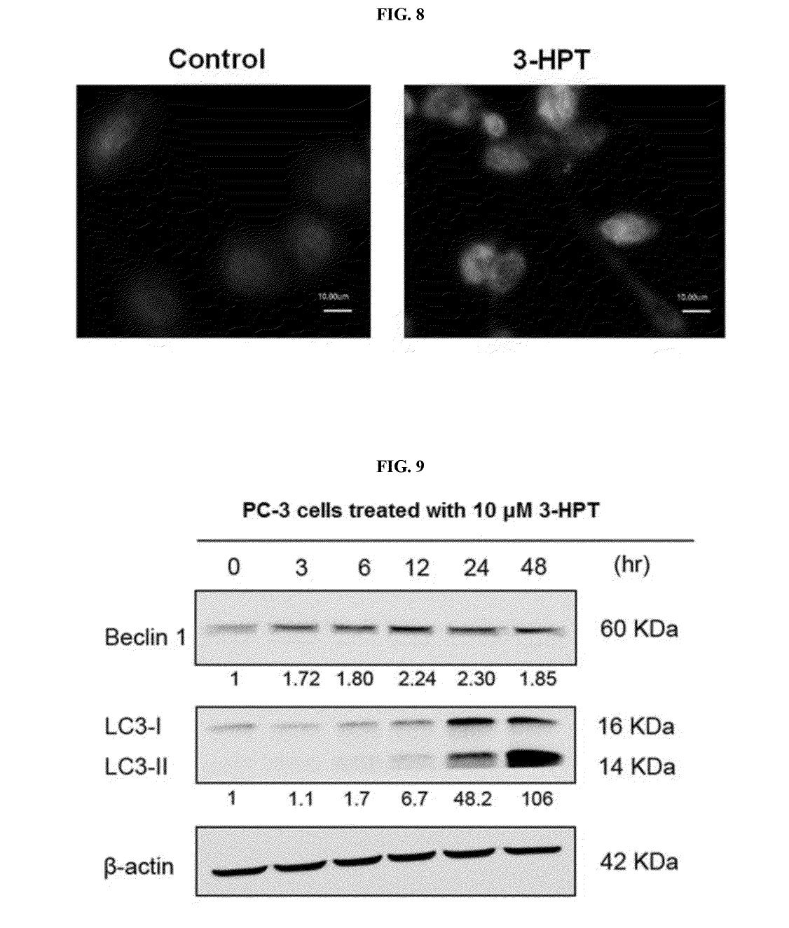

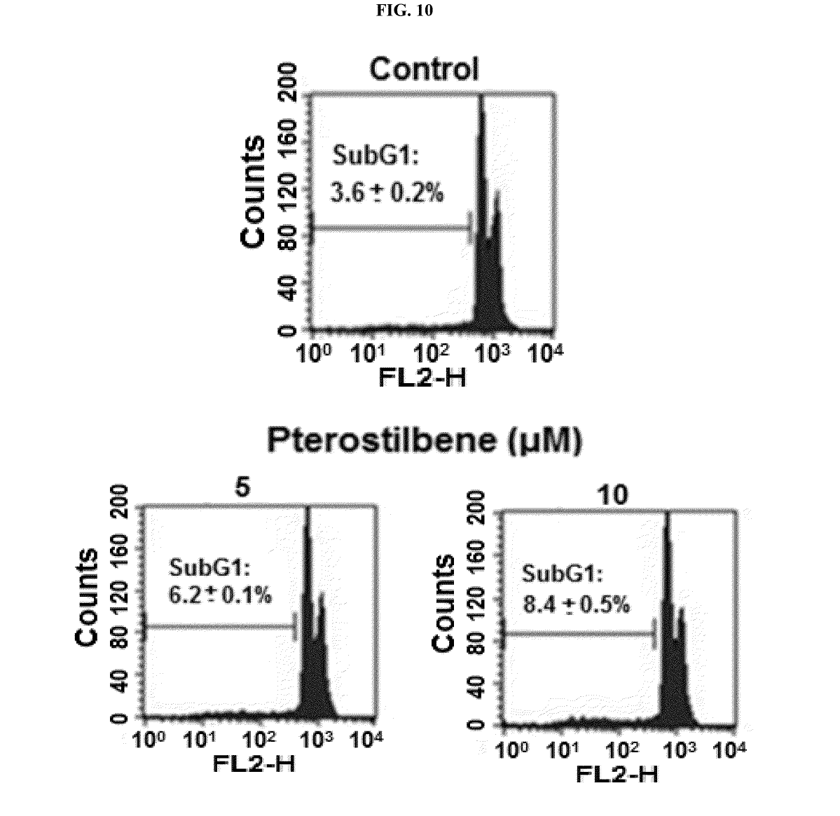

FIG. 8 shows the micrographs of autophagosome formation in PC-3 cells treated with 3-HPT and transfected with the green fluorescence protein-LC3 (GFP-LC3). Photographs were obtained with a fluorescence microscope (Axioscop, Carl Zeiss, Thomwood, N.Y.) equipped with a mercury 100 W lamp, 490 nm band pass blue excitation filters, a 500 nm dichroic mirror and a 515 nm long pass barrier filter. The autophagy induced by 3-HPT in PC-3 cells was also further confirmed by the Western Blotting analysis of Beclin-1, LC3-I, LC3-II, Actin protein expression in PC-3 cells after 10 μM 3-HPT treatment at different time intervals (FIG. 9). The following conclusions were drawn based on aforesaid experimental studies.
1. 3-Hydroxypterostilbene had strong growth inhibitory effect on PC-3 human prostate cancer cells.
2. 3-Hydroxypterostilbene induced the intrinsic apoptotic pathway and extrinsic apoptotic pathway in PC-3 cells.
3. 3-Hydroxypterostilbene also induced autophagic cell death by increasing the Beclin-1, LC3 protein expression and autophagosome formation.
4. 3′-Hydroxypterostilbene has significant therapeutic potential for the treatment of human prostate cancer.

While the invention has been described with reference to a preferred embodiment, it is to be clearly understood by those skilled in the art that the invention is not limited thereto. Rather, the scope of the invention is to be interpreted only in conjunction with the appended claims.

We claim:

1. A method of treating mammals suffering from colorectal carcinoma (tumor) by administering 3,4-dihydroxy-3′,5′-dimethoxy-trans-stilbene(3-hydroxypterostilbene) wherein said method comprises step of bringing into contact effective concentration of 3,4-dihydroxy-3′,5′-dimethoxy-trans-stilbene(3-hydroxypterostilbene) and colorectal carcinoma cells to bring about the effects of reduced tumor volume and tumor weight along with down-regulation of tumor promoting proteins.

2. The method according to claim 1 wherein the colorectal carcinoma is human colorectal carcinoma.

3. The method according to claim 1 wherein said mammal is human.

4. The method according to claim 1 wherein step of administration is one selected from the group consisting of oral administration, intra-peritoneal administration, intra muscular and intra venous administration.

5. A method of inhibiting proliferation of human colon cancer cells using 3,4-dihydroxy-3′,5′-dimethoxy-trans-stilbene(3-hydroxypterostilbene), said method comprising steps of
a. Plating human colon cancer cells like COLO 205, HCT-116 and HT-29 at a density of 2×10⁵ cells/mL into 96 well plates;
b. Treating the cells of step a. following overnight growth to series of graded concentrations of 3-hydroxypterostilbene for 24 hours;
c. Adding 0.2% 3(4,5dimethylthiazol-2-yl)2,5diphenyltetrazolium bromide (MTT) to the cells of step b followed by further incubation of 4 hours; and
d. Measuring cell viability of MTT treated cells of step c using ELISA reader with 570 nm filter.

6. A method of treating human prostate cancer using effective concentration of 3,4-dihydroxy-3′,5′-dimethoxy-trans-stilbene(3-hydroxypterostilbene) wherein said method comprises step of bringing into contact effective concentration of 3,4-dihydroxy-3′,5′-dimethoxy-trans-stilbene(3-hydroxypterostilbene) and human prostate cancer cell.
